(19) United States
(12) Patent Application Publication
(10) Pub. No.: US 2007/0093853 A1
(43) Pub. Date: Apr. 26, 2007

(54) Multi-tweezer hair removal apparatus and method

(75) Inventor: Yehuda Poran, Kibbutz El-Rom (IL)

Correspondence Address: STEIN, MCEWEN & BUI, LLP, 1400 EYE STREET, NW, SUITE 300, WASHINGTON, DC, 20005, US

(73) Assignee: Epilady 2000, L.L.C, Dover, DE (US)

(21) Appl. No.: 11/258,013

(22) Filed: Oct. 26, 2005

(51) Int. Cl. A61B 17/50

(52) U.S. Cl. 606/133

(57) ABSTRACT

An epilating device to remove hair, including a frame including a power source frame, a reduction gear system frame, and a base, a power source supported in the power source frame to provide power to the hair removing operation, a reduction gear system supported in the reduction gear frame to transfer power from the power source to the base, and an epilating unit supported by the base. The epilating unit includes a plurality of epilators tightly aligned in a column along a curved shaft, each epilator having a set of tweezers disposed with respective tweezing ends radially extending to be in line with an outer wall of the column, to rotate about the curved shaft according to the power transferred by the reduction gear system. A curve in the curved shaft induces the tweezers to close when the tweezers pass along the outside of the curve during the rotation.

### BACKGROUND OF THE INVENTION

An aspect of the present invention relates to a hair removal apparatus and method, and, more particularly, to a hair removing apparatus and method using an epilating device.

### DESCRIPTION OF THE RELATED ART

In conventional hair removal devices there are generally one of two methods for removing hair available. According to the first method, the hair is cut, leaving the roots intact beneath the skin surface. According to the second method, sometimes referred to as epilation, hair is removed by pulling individual strands of hair out from their roots.

Several of the proposed hair removal systems involve the use of tilted discs which come together at a point to grasp one or more strands of hair. Other mechanisms involve the use of cams to alternately bring the discs together and apart, thereby trapping strands of hair. Various such mechanisms are discussed in U.S. Pat. No. 4,935,024 to Dolev, U.S. Pat. No. 5,057,115 to Dolev, U.S. Pat. No. 5,190,559 to Gabion, et al, U.S. Pat. No. 5,797,925 to Heintke, U.S. Pat. No. 5,857,903 to Ramspeck, et al, U.S. Pat. No. 5,312,419 to Garenfeld, et al, U.S. Pat. No. 5,196,021 to Kabla, U.S. Pat. No. 5,281,233 to Dolev, and U.S. Pat. No. 5,462,557 to Jordan, et al.

One well known type of hair removal device operated by the epilation method are epilating devices (hereinafter referred to as “epilating devices”). Often, the devices may be considered disc mechanisms because they include a plurality of discs and associated tweezers for each disc. When the tweezers are closed, hair is trapped. The discs, which rotate, produce a torque as a result of the rotation and uproot the hair trapped by the tweezers. Further, the plurality of the discs is usually arranged to have a cylindrical shape.

However, these disc mechanisms may be subject to constraints. The tweezers associated with each disc must close quickly. The pressure exerted by each tweezer must be neither too great nor too little. In the former case where the pressure is too great, the hair would be cut. In the latter case where the pressure is too little, the hair would slide through without being pulled out at its roots. Lastly, the contacting mechanism must be simple, operate reliably over time, and be easy to maintain.

A side view of a conventional cylindrical epilating device is shown in FIG. 1. According to FIG. 1, a series of discs including thin tweezing arms arranged with a series of blocks interposed between the tweezing arms are aligned along a shaft. Here, it may be seen that the external force P on the outer discs causes a compression of the discs such that a force P is applied to each tweezer (a combination of an edge of a tweezing arm and an edge of a block) along the shaft. However, in order to obtain the optimal angle for a tweezing operation, as shown in FIG. 1, the number of tweezers must be limited.

A side view of another type of cylindrical epilating device is shown in FIG. 2. Here, the tweezers move together to close a fixed space D. The cumulative force required to accomplish this is equal to the force required to close each tweezer (P) times the number of tweezers (3) plus an additional force to compensate for precision errors. Thus, the tweezing force required in this epilating device is at least 3P.

Other epilating devices use disc mechanisms employing a large spring with bearings connected to its ends. In such devices, the spring presses on the bearings producing a constant force which acts identically over all the discs and their associated tweezers. The magnitude of the force is the same throughout the entire mechanism. The forces and energy required to operate such mechanisms is relatively low. However, due to technical limitations, the number of tweezers must be kept relatively low.

Thus, there is a need for an epilating device that is easy and inexpensive to assemble and to maintain and which can uproot a greater number of hairs over a larger area than is possible using conventional devices. In addition, there is also an ongoing need for an epilating device that reduces discomfort associated with hair removal.

### SUMMARY OF THE INVENTION

According to an aspect of the present invention, an epilating device in provided in which tweezers of a curved column of epilating discs are closed at an outside of the curve of the column by a force applied to an inside of the curve of the column.

According to an aspect of the present invention, an epilating device in provided in which relatively low forces and less production accuracy are required for operation while a relatively large number of tweezers may be employed.

According to an aspect of the invention, an epilating device to remove hair, comprises a frame including a power source frame, a reduction gear system frame, and a base; a power source supported in the power source frame to provide power to the hair removing operation; a reduction gear system supported in the reduction gear frame to transfer power from the power source to the base; and an epilating unit supported by the base. The epilating unit includes a plurality of epilators tightly aligned in a column along a curved shaft, each epilator having a set of tweezers disposed with respective tweezing ends radially extending to be in line with an outer wall of the column, to rotate about the curved shaft according to the power transferred by the reduction gear system. A curve in the curved shaft induces the tweezers to close when the tweezers pass along the outside of the curve during the rotation.

According to another embodiment, the invention may be understood as a device to perform a hair removal operation. The device comprises a central base, a pair of rigid outer members disposed at opposite ends of the base to extend in substantially similar directions, each outer member including an outer tweezing surface at a distal end thereof; and a pair of inner members suspended along the base between the respective outer members and at a distance from each other. First portions of the inner members extend from the base in parallel with the outer members and second portions of the inner members extend from the base in reverse directions from that of the first portions. Each first portion includes an inner tweezing surface. According to this embodiment, upon an application of a force compressing the second portions of the inner members together, the inner members each pivot about a portion of the base between the inner members, and the inner tweezing surfaces of the first portions are forced to move towards the outer tweezing surfaces of the adjacent outer members.

According to another aspect of the invention, a method of removing hair from a portion of skin of a user is provided. The method comprises positioning an epilating unit, including a plurality of basic epilators tightly aligned in a curved column rotateable about a central curved axis thereof, proximate to the portion of skin, the basic epilators each having a set tweezers, which each face a side of the column, that close as the side of the column rotates toward an outside of the curve and that open as the side of the column rotates toward an inside of the curve, stretching the portion of skin by pressing a side of the column corresponding to an outside of the curve into the portion of the skin, and rotating the column such that the closing tweezers trap and pull the hairs to be removed out of the portion of skin and then open.

Additional and/or other aspects and advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a schematic diagram of an epilating device according to the prior art;

FIG. 2 is a schematic diagram of an epilating device according to the prior art;

FIG. 3A is a perspective view of an epilating device according to an embodiment of the present invention;

FIG. 3B is a perspective view of separated features of the epilating device of FIG. 3A;

FIG. 4 is a side view of the epilating unit according to the embodiment illustrated in FIGS. 3A and 3B;

FIG. 5 is a perspective view of a basic epilator according to an embodiment of the invention;

FIG. 6 is a perspective view of a disassembled basic epilator according to an embodiment of the present invention;

FIGS. 7A and 7B are schematic diagrams of an epilating unit according to an embodiment of the present invention;

FIG. 8 is a plan view of an epilating unit according to an embodiment of the present invention;

FIG. 9 is a plan view of a device to perform a hair removal operation according to another embodiment of the invention; and

FIG. 10 is a diagram of a method of removing hair according to an embodiment of the invention.

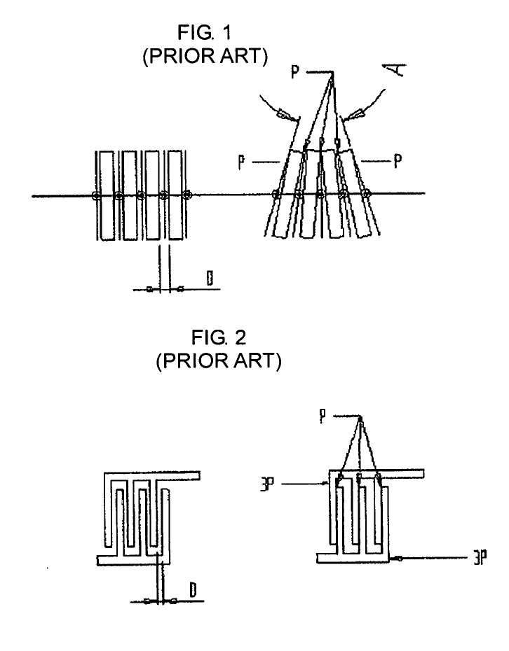

### DETAILED DESCRIPTION OF THE EMBODIMENTS

Reference will now be made in detail to the present embodiments of the present invention, examples of which are illustrated in the accompanying drawings, wherein like reference numerals refer to the like elements throughout. The embodiments are described below in order to explain the present invention by referring to the figures.

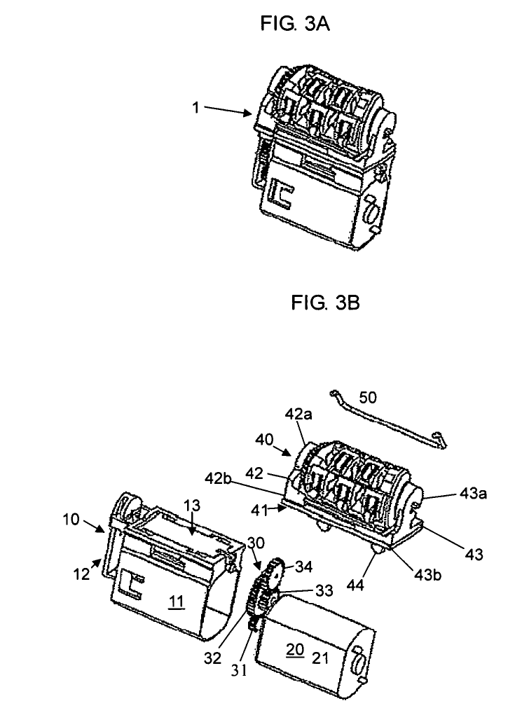

With reference to FIGS. 3A and 3B, an epilating device 1 according to an embodiment of the present invention includes a frame 10, a power source 20, a reduction gear system 30, an epilating unit 40, and a spring 50. The frame 10 includes a power source frame 11, a reduction gear frame 12, and a base 13. The power source frame 11 and the reduction gear frame 12 each define an interior that is of sufficient size and shape to tightly fit around the power source 20 and the reduction gear system 30, respectively. The base 13 rests at one end of the power source frame 11 and communicates with both the power source frame 11 and the reduction gear frame 12.

In an embodiment of the invention, the power source 20 includes a motor 21. As illustrated in the embodiment shown in FIG. 3B, the motor 21 is rectangular with two opposing sides protruding cylindrically. Therefore, with the power source frame 11 tightly fit around the motor 21, the epilating device achieves a size and shape appropriate for intended use thereof. Of course, it is understood that the motor 21 could be replaced with another power source and that whatever power source is chosen may have various shapes.

The reduction gear system 30 includes a system of gears to transfer inputted rotational power from the power source 20 to the epilating unit 40. In an embodiment of the invention, the gears include a first gear 31 attached to a rotating axle (hidden from view) of the power source, a second gear 32 coupled to the first gear, a third gear 33 coaxial with the second gear 32 and having a smaller diameter than the second gear 32, and a fourth gear 34 to transfer the rotational power from the third gear toward the base 13 where the rotational power is applied to the epilating unit 40.

According to an embodiment of the invention, the epilating unit 40 includes a movable unit 41 that allows the epilating unit 40 to be removably installed onto the base 13 of the frame 10. The movable unit 41 includes a first part 42, a second part 43, and attachment parts 44.

The first part 42 includes a flat part to which a first bookend piece 42a is attached. The bookend piece 42a provides a slot into which a first end of a curved shaft 45 (to be discussed later) is inserted and a smooth face upon which a gear bearing holder 46 (to be discussed later) of the epilating unit 40 may rotate. The first part 42 of the movable unit 41 further includes wings 42b disposed along a surface of the flat part opposite the surface to face the power source frame 11. The second part 43 includes a second bookend piece 43a that provides a slot into a second end of the curved shaft is inserted and a smooth face upon which a bearing holder 47 (to be discussed later) of the epilating unit 40 may rotate. The second part 43 of the movable unit 41 further includes extensions 43b extending from the second bookend piece 43a and tightly fit between the wings 42b and the flat part of the first part 42 such that the slots of the first and second bookend pieces 42a and 43a are substantially opposite one another and so that the first and second bookend pieces 42a and 43a remain in that condition until a user detaches them from each other. The attachment parts 44 are disposed on the surface of the flat part to be adjacent to the power source frame 11 and cooperate with the base 13 to hold the epilating unit in place.

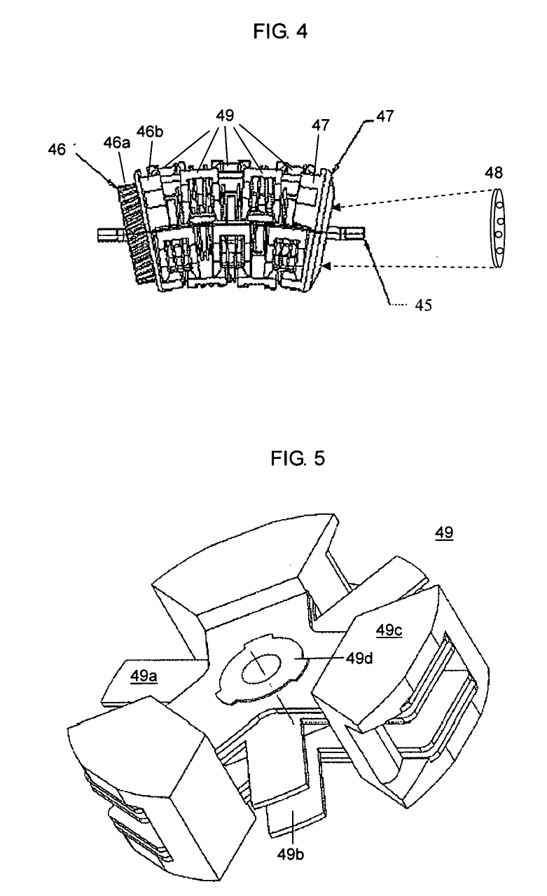

With reference to FIGS. 4 through 6, the hair removing parts of the epilating unit 40 will now be described. As shown in FIG. 4, the curved shaft 45 is, according to an embodiment of the invention, a metallic shaft with flattened ends that fit into the slots of the first and second bookend pieces 42a and 43a. Due to the cooperation of the flattened ends and the slots, the curved shaft 45 is held in a stationary position once the various features are installed together.

The gear bearing holder 46 and the bearing holder 47 are situated just inside the first and second bookend pieces 42a and 43a, respectively. The gear bearing holder 46 includes a gear 46a that is coupled to the fourth gear 34 of the gear reduction system 30. The bearing holder 47 includes a circular groove into which a plate 48 fitted with ball bearings is tightly fit. Both the gear bearing holder 46 and the bearing holder 47 include a center opening through which the curved shaft 45 extends. Both the gear bearing holder 46 and the bearing holder 47 further include protrusions 46b and 47b that, according to an embodiment of the invention, are disposed evenly around inside surfaces of the gear bearing holder 46 and the bearing holder 47 in groups of three.

A set of basic epilators 49, illustrated in FIG. 4, is then arranged to be held along the curved shaft 45 between the gear bearing holder 46 and the bearing holder 47. As shown in FIG. 5 and as will be discussed below, each basic epilator is formed of a pair of inner metal parts 49a, a pair of outer metal parts 49b, an outer lock 49c, and a hub 49d. Briefly, the metal parts are held together around the hub 49d by the outer locks 49c, which are disposed in such a way as to fit onto other basic epilators as well as the protrusions 46b and 47b of the gear bearing holder 46 and the bearing holder 47. As a result, when assembled along the curved shaft 45, the basic epilators 49 fit into a staggered formation.

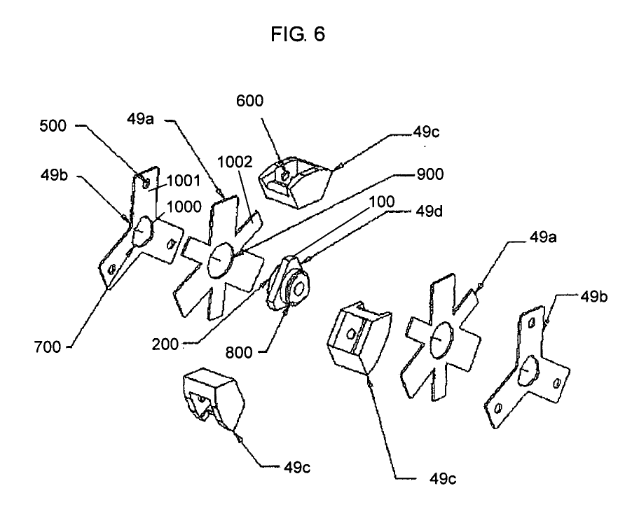

With reference to FIG. 6, the structure of the basic epilator will now be described in detail. The hub 49d is made of plastic or other similar materials and includes a triangular central portion 100 and a pair of cylinders 200 protruding from each side of the triangular central portion 100. Each cylinder 100 includes an end portion having protrusions 800 protruding therefrom.

The inner and outer metal parts 49a and 49b, on the other hand, are metallic and have holes 900 through their respective central portions 1000. The outer metal parts 49b also include notches 700 defined in the edges of their holes 900. Both the inner and outer metal parts 49a and 49b have a flattened tripod-like structure where three arms 1001 extend from the central portions 1000, although the inner metal parts 49a further include three secondary arms 1002 extending from the central portions 1000 between each of the arms 1001 in a secondary tripod-like structure. According to an embodiment of the invention, the arms 1001 are wider than the secondary arms 1002.

Initially, each inner metal part 49a is mounted around the triangular central portion 100 of the hub 49d from each side by inserting the cylinders 200 through the holes 900 in each inner metal part 49a such that the arms 1001 line up with and appear to extend from the faces of the triangular central portion 100. Then, each outer metal part 49b is assembled onto the cylinders at a distance from each inner metal part 49a by fitting the notches 700 over the protrusions 800. Three outer locks 49c are then fitted over each of the three arms 1001 of each of the inner and outer metal parts 49a and 49b. A protrusion 600 of each outer lock 49c cooperates with a hole 500 of each arm 1001 of the outer metal parts 49b to secure the outer locks 49c.

According to an embodiment of the invention, the outer locks 49c are formed of a hard plastic or similar material. As such, the outer locks 49c do not tend to deform under the forces applied during the normal operation of the epilating device.

Similarly, according to an embodiment of the invention, the hub 49d is formed of a hard plastic or similar material. As such, the hub 49d does not tend to deform under the forces applied during the normal operation of the epilating device.

Therefore, when each basic epilator 49 is fully assembled, the basic epilator 49 resembles a two-layer disc, with each disc including an inner metal part 49a and an outer metal part 49b held along the cylinders 200 by the outer locks 49c. The distance between the inner metal parts 49a and the outer metal parts 49b is maintained so as to provide the inner metal parts 49a room to pivot during the hair removing operation. Indeed, each inner metal part 49a is able to pivot about edges of the faces of the triangular central portion 100 when an external force is applied to the secondary arms 1002.

It is understood that in an embodiment of the invention, five basic epilators 49 are assembled between the first and second parts 42 and 43 of the movable unit 41. However, more or less basic epilators 49 may be added as is necessary. It is further understood that while the basic epilators 49, the gear bearing holder 46, and the bearing holder 47 have been described as having a tripod-like format, other configurations are possible.

Once the basic epilators 49 are arranged together along the curved shaft 50 and the first and second parts 42 and 43 of the movable unit 41 are combined, a spring 50 is affixed to grooves defined on respective outer surfaces of the first and second parts 42 and 43. The spring 50 serves to hold the various features together during attachment to the frame 10 and operation of the epilating device.

Therefore, as illustrated in FIGS. 3B and 4, it may be seen that, due to the curvature of the curved shaft 45 and the pressure of the spring 50, the epilating unit 40, including the first and second bookend pieces parts 42a and 43a and the five basic epilators 49 are arranged so as to be in a curved horizontal column, as shown in FIGS. 3B and 4. In other words, one side of the column is compressed and has a longitudinal length L1 while the opposite side of the column is extended and has a longitudinal length L2, where L2 is longer than L1.

It may also be seen that each of the three protrusions 46b and 47b of the gear bearing holder 46 and the bearing holder 47 contact the outer secondary arms 1002 of the basic epilators 49 situated adjacent to each of the gear bearing holder 46 and the bearing holder 47 (hereinafter referred to as “first stage basic epilators 49,” whereas the basic epilators 49 that are adjacent to the first stage basic epilators 49 are hereinafter referred to as “second stage basic epilators 49,” and so forth). Spaces between the protrusions 46b and 47b are loosely occupied by the outer locks 49c of the first stage basic epilators 49.

Similarly, the inner secondary arms 1002 of the first stage basic epilators 49 are contacted by the outer locks 49c of the second stage basic epilators 49 and the outer locks 49c of the first stage basic epilators 49 are contacted by the outer secondary arms 1002 of the second stage basic epilators 49. Meanwhile, in the embodiment of the invention in which five basic epilators 49 are stacked in the column, the inner secondary arms 1002 of the second stage basic epilators 49 are contacted by the outer locks 49c of the third stage basic epilator 49, of which there would only be one, and the outer locks 49c of the second stage basic epilators 49 are contacted by the secondary arms 1002 of the third stage basic epilator 49.

As a consequence of the structure discussed above, depending on the orientation of the column with respect to the curve of the curved shaft 45, some of the arms 1001 of the basic epilators 49, will be either closed in a tweezing position or nearly closed in a hair gripping position. Conversely, the arms 1001 on the opposite side of the basic epilators will be either completely opened or slightly opened in a hair releasing position. Indeed, due to the structure discussed above, where the outer locks 49c of the basic epilators contact the secondary arms 1002, those arms 1001 that are completely closed or nearly closed in a hair gripping position will be located on the outside of the curve of the column.

In greater detail, because each of the basic epilators 49 has the flattened tripod-like structure discussed above, in which the secondary arms 1002 extending from the central portions 1000 in an opposite direction to that of a corresponding set of arms 1001 on the opposite side of the central portion 1000, when the curve of the curved shaft 45 causes the secondary arms 1001 to be compressed toward one another, a corresponding set of arms 1001 on the opposite side of the central portion 1000 are forced into tweezing positions, in which the arms 1001 are compressed toward one another.

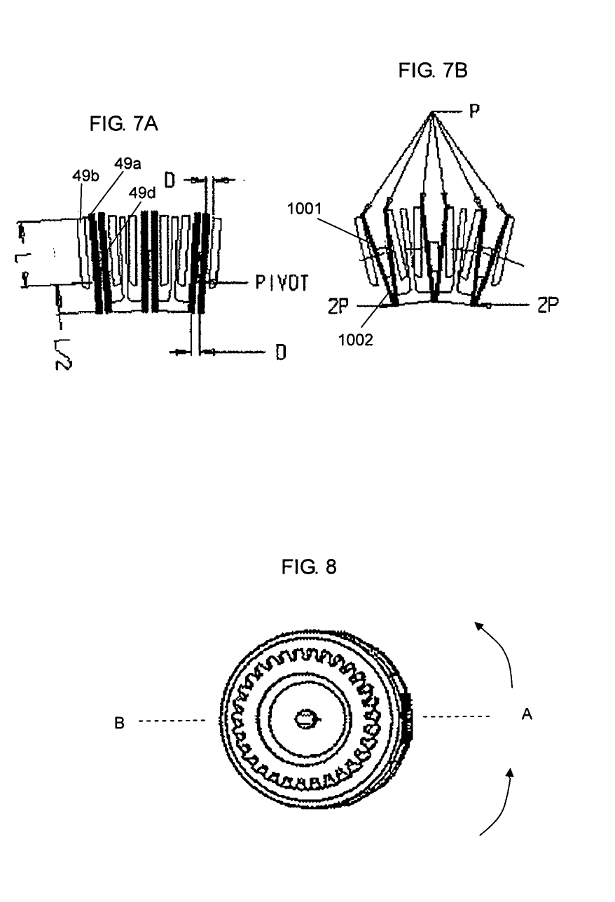

As illustrated in FIGS. 7A and 7B, the structures discussed above enable less force to be required to operate the epilating unit 40. According to FIG. 7A, which is a view of the epilating unit 40 of when the basic epilators 49 are not compressed or expanded (i.e. the portion of the column that are neither inside or outside the curve of the column), a length of the arms 1001 of the inner metal parts 49a from the edge of the hub 49d about which the inner metal part pivots is L whereas a length of the secondary arms is L/2. This ratio of the lengths of the arms 1001 and the secondary arms 1002 provides that while the distance traveled by the inside and outside edges of the inner metal parts 49a is substantially a constant D, the number of spaces across which the outside edges of the inner metal parts 49a travels is double the number of spaces across which the outside edges of the inner metal parts 49a travels.

Further, as illustrated in FIG. 7B, it follows that a force 2P that is applied to the three sets of inside edges of the inner metal parts 49a (i.e. by the epilating unit rotating towards the inside of the curve of the curved shaft 45) is distributed to the six outer edges of the inner metal parts 49a so as to press the six outer edges of the inner metal parts 49a against the outer metal parts 49b with a force P. In addition, since the force 2P is divided between the sets of inner metal parts 49a, high production accuracy is not required.

The operation of the epilating device 1 will now be described with reference to FIGS. 3B and 4. As illustrated in FIGS. 3B and 4, the embodiment of the invention discussed hereinbelow includes 5 basic epilators 49 arranged in a column about a curved shaft 45 and each basic epilator has the tripod structure discussed above. However, more or less basic epilators 49 with different arms structures may be employed.

When the power source 20 is not activated, one side of the column is compressed and has a longitudinal length L1 while the opposite side of the column is extended and has a longitudinal length L2, where L2 is longer than L1. This is the starting position and is determined based the orientation of the column when the power source was previously turned off.

For the purpose of demonstration it will be understood that FIG. 8 presents a view of the column at a starting position in which a series of outer locks 49c of the first stage basic epilators 49 and the third stage basic epilators 49 are disposed along the outside of the curvature of the column at circumferential point A. From this, it may be understood that the secondary arms 1002 of the second stage basic epilators 49 are compressed toward one another by the outer locks 49c of the first and third stage basic epilators 49c at circumferential point B and, as a result, the arms 1001 locked by the outer locks 49c are closed into tweezing positions at circumferential point A.

Once the power source 20 is activated, the power source 20 causes the reduction gear system 30 to rotate. The rotation of the reduction gear system 30 is then transferred to the epilating unit 40 through the interface between the fourth gear 34 and the gear bearing holder 46. Since the protrusions 46b of the gear bearing holder 46 and the bearing holder 47 and the outer locks 49c of the basic epilating units 49 tightly fit together, the epilating unit 40, including the gear bearing holder 46, the basic epilating units 49 and the bearing holder 47, rotates about the curved shaft 45 as a unit.

Since the secondary arms 1001 were already in tweezing positions when the power source was activated, it will be understood that these secondary arms would not have hair caught between them. Thus, substantially no hair removal will occur in the initial rotation of the epilating unit 40.

After approximately 50-60 degrees of rotation, the outer locks 49c of the first and third stage basic epilators will no longer compress the secondary arms 1002 of the second stage basic epilators 49. However, at this point, the protrusions 46b and 47b of the gear bearing holder 46 and the bearing holder 47, respectively, along with the outer locks 49c of the second stage basic epilators will begin to compress the respective secondary arms 1002 of the first and third basic epilators 49 toward each other. This will result in the arms 1001 of the first and third stage basic epilators 49 being forced to close into tweezing positions. As such, any hair caught between the arms 1001 of the first and third stage basic epilators 49 will be caught once the continuing rotation of the epilating unit 40 causes the arms 1001 to completely close into tweezing positions.

With the arms 1001 of the first and third stage basic epilators 49 in tweezing positions, once the epilating unit 40 has rotated approximately 25-30 degrees, or to the point where the secondary arms of the first and third stage basic epilators 49 remain compressed by the protrusions 46b and 47b of the gear bearing holder 46 and the bearing holder 47, respectively, along with the outer locks 49c of the second stage basic epilators 49, the epilating unit 40 will have generated a torque on the hairs that were grabbed by the arms 1001. This torque should be sufficient to pull these hairs from their respective roots.

As the epilating unit 40 continues to rotate, the protrusions 46b and 47b of the gear bearing holder 46 and the bearing holder 47, respectively, along with the outer locks 49c of the second stage basic epilators will no longer compress the secondary arms 1002 of the first and third stage basic epilators 49. As a result, the arms 1001 of the first and third stage basic epilators 49 will open and release any removed hairs.

Meanwhile, the continually rotating epilating unit 40 will approach once again the position illustrated in FIG. 8, albeit nearly 120 degrees of rotation later. Here, the outer locks 49c of the first and third stage basic epilators 49 will begin to compress the secondary arms 1002 of the second stage basic epilators 49. This will result in the arms 1001 of the second stage basic epilators 49 to being forced to close into tweezing positions. As such, any hair caught between the arms 1001 of the second stage basic epilators 49 will be caught once the continuing rotation of the epilating unit 40 causes the arms 1001 to completely close into tweezing positions.

With the arms 1001 of the second stage basic epilators 49 in tweezing positions, once the epilating unit 40 has rotated approximately 25-30 additional degrees, or to the point where the secondary arms of the second stage basic epilators 49 remain compressed by the outer locks 49c of the first and third stage basic epilators 49, the epilating unit will have generated a torque on the hairs that were grabbed by the arms 1001. This torque should be sufficient to pull these hairs from their respective roots.

This process may be continued until the user has determined that a sufficient number of hairs have been removed from their respective roots. It will be understood that the user is responsible for positioning the epilating unit a various positions along the skin so as to maximize the hair removal operation.

Further, it will be understood that since the hair removal operation of the epilating unit 40 occurs generally on the outer side of the curve of the epilating unit 40, as the user moves the epilating unit along the skin from which hair is to be removed, the curve of the epilating unit will naturally stretch the surface of the skin. This may result in an additional benefit in that removing hair from stretched skin is relatively easy.

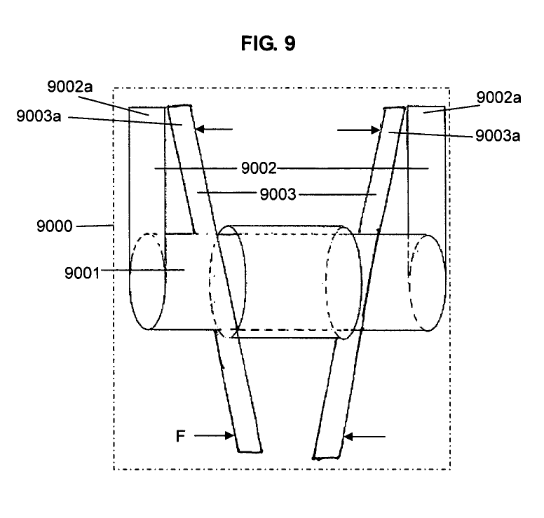

According to another embodiment, the invention may be understood as a device to perform a hair removal operation. As shown in FIG. 9, the device 9000 comprises a central base 9001, a pair of rigid outer members 9002 disposed at opposite ends of the base to extend in substantially similar directions, each outer member including an outer tweezing surface 9002a at a distal end thereof; and a pair of inner members 9003 suspended along the base 9001 between the respective outer members and at a distance from each other. First portions of the inner members extend from the base in parallel with the outer members and second portions of the inner members extend from the base in reverse directions from that of the first portions. Each first portion includes an inner tweezing surface 9003a. According to this embodiment, upon an application of a force F compressing the second portions of the inner members together, the inner members each pivot about a portion of the base between the inner members, and the inner tweezing surfaces of the first portions are forced to move towards the outer tweezing surfaces of the adjacent outer members.

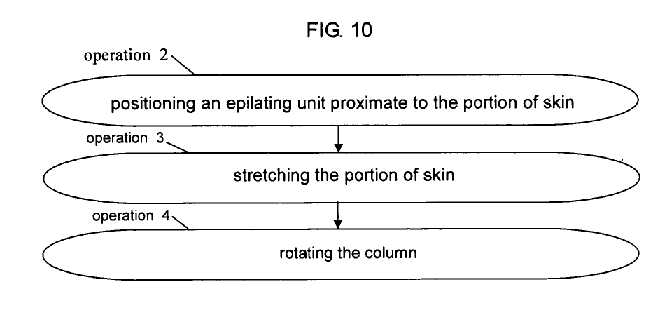

According to another embodiment, the invention may be understood as a method of removing hair from a portion of skin of a user. As shown in FIG. 10, the method comprises positioning an epilating unit, including a plurality of basic epilators tightly aligned in a curved column rotateable about a central curved axis thereof, proximate to the portion of skin, the basic epilators each having a set tweezers, which each face a side of the column, that close as the side of the column rotates toward an outside of the curve and that open as the side of the column rotates toward an inside of the curve (operation 2), stretching the portion of skin by pressing a side of the column corresponding to an outside of the curve into the portion of the skin (operation 3), and rotating the column such that the closing tweezers trap and pull the hairs to be removed out of the portion of skin and then open (operation 4).

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. An epilating device to perform a hair removing operation, comprising:
a frame including a power source frame, a reduction gear system frame, and a base;
a power source supported in the power source frame to provide power to the hair removing operation;
a reduction gear system supported in the reduction gear frame to transfer power from the power source to the base; and
an epilating unit supported by the base, including a plurality of basic epilators tightly aligned in a column along a curved shaft, each epilator having a set of tweezers disposed with respective tweezing ends radially extending to be in line with an outer wall of the column, to rotate about the curved shaft according to the power transferred by the reduction gear system, a curve in the curved shaft inducing the tweezers to close when the tweezers pass along the outside of the curve during the rotation.

2. The device according to claim 1, further comprising a spring to squeeze the plurality of basic epilators together so as to cause the epilators to be tightly aligned.

3. The device according to claim 1, wherein the power source frame and the reduction gear frame each define an interior to tightly fit around the power source and the reduction gear system, respectively, and wherein the base rests at one end of the power source frame to communicate with both the power source frame and the reduction gear frame.

4. The device according to claim 1, wherein the power source comprises an electric motor.

5. The device according to claim 1, wherein the reduction gear system includes a system of gears to transfer rotational power from the power source to the epilating unit.

6. The device according to claim 5, wherein the gears include a first gear attached to the power source, a second gear coupled to the first gear, a third gear coaxial with the second gear and having a smaller diameter than the second gear, and a fourth gear to transfer the rotational power from the third gear toward the base.

7. The device according to claim 1, wherein the epilating unit comprises a movable unit that allows the epilating unit to be removably installed onto the base.

8. The device according to claim 7, wherein the movable unit comprises:
a first part, including a flat part to which a first bookend piece is attached, the first bookend piece having a slot into which a first end of the curved shaft is inserted, and wings disposed along a surface of the flat part;
a second part, including a second bookend piece having a slot into which a second end of the curved shaft is inserted, and extensions to tightly fit between the wings and the flat part of the first part such that the slots of the first and second bookend pieces are substantially opposite one another; and
attachment parts disposed on the surface of the flat part to cooperate with the base.

9. The device according to claim 8, wherein the first and second bookend pieces each comprise a smooth face across which outer ends of the epilating unit slides.

10. The device according to claim 8, wherein the curved shaft comprises a metallic shaft with flattened ends that fit into the slots of the first and second bookend pieces.

11. The device according to claim 8, wherein the epilating unit comprises:
a gear bearing holder just inside the first bookend piece including a gear to be coupled to the gear reduction system; and
a bearing holder just inside the second bookend piece including a circular groove into which a plate fitted with ball bearings is tightly fit, wherein both the gear bearing holder and the bearing holder comprise:
a center opening through which the curved shaft extends; and
protrusions disposed evenly around and from respective inside surfaces thereof.

12. The device according to claim 11, wherein the basic epilators are aligned along the curved shaft between the gear bearing holder and the bearing holder.

13. The device according to claim 12, wherein the tweezers of each basic epilator comprise a hub and a pair of inner metal parts respectively coupled with but separated from outer metal parts held together on opposing sides of the hub by a set of outer locks, the outer locks being disposed in a pattern so as to fit next to the pattern of outer locks of other basic epilators as well as the protrusions of the gear bearing holder and the bearing holder.

14. The device according to claim 13, wherein the hub and the outer locks comprise plastic or other similar materials and the inner and outer metal parts comprise metallic materials.

15. The device according to claim 13, wherein the hub comprises a triangular central portion and a pair of cylinders protruding from each side of the triangular central portion, each cylinder including an end portion having protrusions protruding radially therefrom.

16. The device according to claim 15, wherein the inner and outer metal parts each comprise:
central portions through which holes are defined to receive the cylinders of the hub; and
three arms to extend from the central portions, wherein the edges of the central portions of the outer metal parts include notches defined therein.

17. The device according to claim 16, wherein the inner metal parts further comprise secondary arms extending from the central portions between each of the arms.

18. The device according to claim 17, wherein each inner metal part is mounted on opposite sides of the triangular central portion with each of the cylinders penetrating through the holes in the inner metal parts such that the arms line up with and the faces of the triangular central portion, and each outer metal part is assembled adjacent to respective outer surfaces of the inner metal parts by fitting the notches of the outer metal parts over the protrusions of the cylinders.

19. The device according to claim 18, wherein the outer locks each fit around corresponding arms of each coupling of the inner and outer metal parts.

20. The device according to claim 18, wherein each inner metal part pivots about an edge of a face of the triangular central portion when an external force is applied to the secondary arms.

21. The device according to claim 20, wherein the plurality of basic epilators comprise five basic epilators assembled between the first and second parts of the movable unit in a curved column, the two outer basic epilators being first stage basic epilators, the central basic epilator being a third stage basic epilator, and the other two epilators being second stage basic epilators.

22. The device according to claim 21, wherein the protrusions of the gear bearing holder and the bearing holder contact the outer secondary arms of the first stage basic epilators, the inner secondary arms of the first stage basic epilators are contacted by the outer locks of the second stage basic epilators while the outer locks of the first stage basic epilators are contacted by the outer secondary arms of the second stage basic epilators, and the inner secondary arms of the second stage basic epilators are contacted by the outer locks of the third stage basic epilator while the outer locks of the second stage basic epilators are contacted by the secondary arms of the third stage basic epilator.

23. The epilating device according to claim 1, wherein the epilating unit stretches a portion of skin from which hair is to be removed during the hair removing operation.

24. An epilating unit including a plurality of epilators tightly aligned in a column along a curved shaft between end pieces having inward facing protrusions, each epilator comprising:
a hub including a triangular central portion; and
a pair of inner metal parts respectively coupled with and separated from outer metal parts on opposing sides of the hub, each metal part including central portions from which three arms extend in directions that are perpendicular to each face of the triangular central portion, each inner metal part further including three secondary arms; and
a set of outer locks, each fitting around corresponding arms of each coupling of the inner and outer metal parts, to be disposed in a pattern so as to fit next to patterns of outer locks of other basic epilators as well as the protrusions of the end pieces, wherein an external force compressing the corresponding secondary arms of the pair of inner metal parts towards each other causes the inner metal parts to pivot about a corresponding face of the triangular central portion so as to force corresponding arms of the inner metal parts into contact with corresponding arms of the outer metal parts.

25. A device to perform a hair removal operation, comprising:
a central base;
a pair of rigid outer members disposed at opposite ends of the base to extend in substantially similar directions, each outer member including an outer tweezing surface at a distal end thereof; and
a pair of inner members suspended along the base between the respective outer members and at a distance from each other, first portions of the inner members extending from the base in parallel with the outer members and second portions of the inner members extending from the base in reverse directions from that of the first portions, each first portion including an inner tweezing surface, wherein
upon an application of a force compressing the second portions of the inner members together, the inner members each pivot about a portion of the base between the inner members, and
the inner tweezing surfaces of the first portions are forced to move towards the outer tweezing surfaces of the adjacent outer members.

26. A method of removing hair from a portion of skin of a user, comprising:
positioning an epilating unit, including a plurality of basic epilators tightly aligned in a curved column rotateable about a central curved axis thereof, proximate to the portion of skin, the basic epilators each having a set tweezers, which each face a side of the column, that close as the side of the column rotates toward an outside of the curve and that open as the side of the column rotates toward an inside of the curve;
pressing a side of the column corresponding to an outside of the curve into the portion of the skin; and
rotating the column such that the closing tweezers trap and pull the hairs to be removed out of the portion of skin and then open.
